(19) United States
(12) Patent Application Publication
(10) Pub. No.: US 2006/0083684 A1
(43) Pub. Date: Apr. 20, 2006

(54) Method of radio-labelling biomolecules

(76) Inventors: Paul Howard Walton, Helsington York (GB); Timothy Smith, Aberdeen (GB)

Correspondence Address: MYERS BIGEL SIBLEY & SAJOVEC, PO BOX 37428, RALEIGH, NC, 27627, US

(21) Appl. No.: 10/504,374

(22) PCT Filed: Feb. 7, 2003
(86) PCT No.: PCT/GB03/00548

§ 371 Date: May 16, 2005

(30) Foreign Application Priority Data
Feb. 12, 2002 (GB) 0203330.6
Jul. 5, 2002 (GB) 0215511.7

(51) Int. Cl. A61K 51/00; C07K 14/79

(52) U.S. Cl. 424/1.69; 530/400

(57) ABSTRACT

A method of radio-labelling a biomolecule comprising contacting the biomolecule with a source of radionuclide, such as technetium, in the presence of a weak transfer ligand and optionally subsequently passing the mixture through a size-exclusion filtration process. Also claimed are kits comprising such novel compositions, especially lactoferrin coupled to chemotherapeutic agents, and uses therefor.

The present invention relates to a method of labelling biomolecules with radionuclides, particularly but not exclusively with technetium, kits comprising ingredients/components to perform the method, novel compositions and uses therefor.

### BACKGROUND TO THE INVENTION

Radio-labelling of biomolecules has been used as a means to track and detect the pathway or location of a particular biomolecule when administered to a patient or subject. Such radio-labelled biomolecules are capable of emitting low levels of radiation, which can be detected and pin-pointed to a target organ or other substrate. Radionuclides such as rhenium-186m and, particularly, technetium-99m, are useful for biomolecule labelling since they are known to form relatively stable bonds with a variety of biomolecules. In quantitative terms, technetium (Tc) compounds are by far the most important radiopharmaceuticals used today with an estimated market share of more than 80%.

For radio-medical purposes, the isotope ⁹⁹Tc is important not in its slowly β-decaying ground state but in a metastable, nuclear excited state, i.e. as exclusively γ-emitting ^{99m}Tc with a diagnostically useful half-life of six hours. One of the major reasons for the popularity of this radioisotope in radio-diagnostics is the availability of an easily operable technetium ‘reactor’ or ‘generator’, which allows the convenient preparation of applicable solutions in a normal clinical environment.

It is known from the prior art to use filtration to prepare radio-labelled compounds. However, one of the problems associated with current labelling methods of biomolecules is that they produce many different technetium species within solution and so lack purity. Since technetium compounds are used to target specific organs in vivo it is desirable that its compounds are as pure as possible before they are introduced into a recipient/patient.

A further problem associated with the prior art methods is that the labelling efficiencies are not high. This leads to considerable waste of expensive materials and increases in preparation time.

It is known from the prior art to use technetium-labelled transferrin as a potential in vivo tumour-imaging agent (Paik et al J Radioanal Chem 1980; 60: 281-289). This study showed that although some uptake of Tc-sTf into tumour cells occurred (0.36% of injected dose) the uptake is poor compared to non-specifically bound activity. Indeed the authors state that Tc-labelled transferrin does not appear to be a suitable imaging agent because of the low tumour to blood ratio post injection. Accordingly technetium labelled transferrin has not been the compound of choice in radio-medical areas but rather transferrin labelled with other radionuclides such as ¹²⁵I, ¹¹¹In and ⁶⁷Ga where labelling efficiency is higher and thus tumour uptake greater.

A method which could improve technetium labelled transferrin efficiency and stability and eventual uptake into tumour cells would offer an immediate advantage to nuclear medicine and diagnostic procedures.

It is an object of the present invention to provide a method for radionuclide-labelling of biomolecules which advantageously removes much of the extraneous radionuclide material, leading to high labelling efficiencies and purer radionuclide-labelled materials than has hitherto been possible using prior art methods.

### STATEMENT OF THE INVENTION

In its broadest aspect the present invention provides a method of radio-labelling a biomolecule comprising contacting the biomolecule with a source of radionuclide in the presence of a transfer ligand and optionally subsequently passing the mixture through a size-exclusion filtration process so as to selectively collect the radio-labelled biomolecule.

According to a first aspect of the invention there is provided a method of radio-labelling a biomolecule comprising contacting the biomolecule with a source of radionuclide in the presence of a weak transfer ligand.

Reference herein to a “biomolecule”, includes any product or composition of matter capable of forming a complex with the radionuclide, for example the biomolecule independently has groups to complex with the radionuclide for example the proteins, polypeptides, monoclonal or polyclonal antibodies or antibody fragments, albumins, drugs, cytokines, enzymes, hormones, immune modulators, receptor proteins and the like. It is to be understood that when the biomolecules to be labeled are antibody fragments, the antibody fragments can be those that bind to antigens which include, but are not limited to, antigens produced by or associated with tumours, infectious lesions, microorganisms, parasites, myocardial infarctions, clots, atherosclerotic plaque, or normal organs or tissues. The term “biomolecule” includes reference to multiple unit proteins (i.e. proteins containing more than one molecule) and less preferably to biological products derivated to add a complexing moiety.

Accordingly the radionuclide-labelling methods of the present invention are useful for the radionuclide labelling of any of the aforementioned biomolecules.

Reference herein to a “transfer ligand” is a ligand that forms an intermediate complex with the radionuclide that is stable enough to prevent unwanted side-reactions but labile enough to be converted to the radio-labelled biomolecule. The formation of the intermediate complex is kinetically favoured while the formation of the radio-labelled biomolecule is thermodynamically favoured. In general, transfer ligands are comprised of oxygen or nitrogen donor atoms.

Preferably, the weak transfer ligand has low stability and is non-chelating ligand.

Preferably the weak exchange ligand has an association constant of between 0.01 dm³ mol⁻¹ and 1000 dm³ mol⁻¹.

Preferably the weak exchange ligand has a low stability constant.

Preferably the weak exchange ligand is selected from the group comprising thiourea, urea or ammonia.

Preferably, the reaction mixture is in solution.

Preferably, the radionuclide source is a ^{99m}Tc source, more preferably the source is pertechnetate i.e. TcO₄⁻. Typically, the pertechnetate source is provided as a solution; typically it is generated at the site where the investigation/treatment is to take place.

Other suitable radionuclides may be selected from the group comprising ⁵⁷Co, ⁶⁷Cu, ⁶⁷Ga, ⁹⁰Y, ⁹⁷Ru, ¹⁶⁹Yb, ¹⁸⁶Re, ¹⁸⁸Re, ²⁰³Pb, ¹⁵³Sm and/or ²¹²Bi.

Preferably, the biomolecule, radionuclide and transfer ligand mixture further includes a reduction step using a reducing agent, the function of the reducing agent being to convert Tc as the pertechnetate (TcO₄⁻) to Tc³⁺ so that it is in a form that may more easily bind to the biomolecule. In this respect the reducing agent may comprise any agent that is capable of performing the reduction step.

Preferably, the reducing agent is selected from the group comprising a tin(I) salt for example chloride, nitrite and/or sulphite. Another preferred reducing agent is ascorbic acid/ascorbate.

Preferably, in the instance of including the size-exclusion step it comprises passing the reaction mixture through a tube and filter system, for example and without limitation, a Centricon filter such as a Centricon 30 filter. Such a filter allows passage of biomolecules of less than 30,000 daltons therethrough whilst trapping biomolecules of more than 30,000 daltons on the filter surface. It will be appreciated that the size of the filter is selected according to the biomolecule of choice and is not intended to limit the scope of the invention.

Preferably, the method further includes the step of double filtration. In this respect the mixture is introduced into tube having a closed end and passed through a filter, the filter being held in a transverse position with respect to the longitudinal tube walls by a frit or the like.

Preferably, the mixture is subsequently or simultaneously centrifuged in an appropriate machine at a speed of, for example, around 2000 to 5000 rpm or more, and more preferably at between 3000 to 4000 rpm and most preferably at around 3200 rpm. Once the solution has passed through the filter and biomolecules of selected size have been excluded i.e. small sized molecules have passed through the filter and reside in solution in the bottom of the tube, they may then be discarded. Biomolecules of a size above the selected exclusion size remain on a upper surface of the filter.

Preferably, the filter is then reversed so that biomolecules above the size of the filter exclusion size may be washed off, typically into the bottom of a tube.

Preferably, the washed off radio-labelled biomolecule is further centrifuged at around 2000 to 4000 rpm, and typically at 2500 rpm so as to collect the radio-labelled biomolecule in the bottom of the tube.

The double filtration process preferably comprises:
(i) introducing the reaction mixture into a tube having an open end and a closed end and being provided with a transverse filter, the filter suitably being held in a transverse position with respect to the longitudinal tube walls by a frit or the like;
(ii) collecting material of a selected size on an upper surface of the filter;
(iii) reversing the filter so that material initially collected on its upper surface is then on a lower surface of the filter; and
(iv) washing the material off said lower surface of the filter and collecting it for example into a closed end of a tube.

The method of the present invention advantageously provides improved purity of radio-labelled, especially technetium labelled, biomolecules and more especially technetium labelled lactoferrin and/or transferrin with a reduced amount of extraneous material. This is achieved by binding of the biomolecule to the weak exchange ligand, typically thiourea, and optionally and subsequently using size exclusion filtration.

Preferably, the method further includes the step of removing any weakly bound radionuclide. This may be achieved by either acid stripping, for example exposure to acid conditions, for example pH 5, or alternatively by competition with a chelating moiety for example and without limitation by mixing with diethylenetriaminepentaacetic acid (DTPA). In this way the concentration of tightly bound radionuclide-labelled biomolecule may be advantageously be further improved.

Preferably, in the instance of the biomolecule having disulphide bonds, it is pre-incubated with a biomolecule reducing agent prior to exposure to the radionuclide. The biomolecule reducing agent reduces disulphide bonds into two sulfhydryl bonds thus increasing access of the biomolecule binding sites to the radionuclide of choice. 2-mercaptoethanol is a suitable biomolecule reducing agent.

Preferably, the pre-incubation step comprises incubating the biomolecule with the biomolecule reducing agent for between 6 to 24 hours.

Preferably, the biomolecule is in holo-form.

Preferably, the concentration of the biomolecule reducing agent is in the region of 2 to 100 μM. We have demonstrated that increasing the concentration of the biomolecule reducing agent in the pre-incubation step increases the labelling efficiency of the biomolecule when exposed to the radionuclide.

According to a yet further aspect of the invention there is provided a kit comprising a biomolecule, a source of radionuclide and a weak transfer ligand and, optionally, a set of written instructions.

Preferably, the kit further includes a biomolecule reducing agent and/or a radionuclide reducing agent.

According to yet further aspect of the invention there is provided a radionuclide-labelled product produced obtainable by, or produced by, the methods of the present invention. The invention includes a radio-labelled product having the characteristics of a product produced by the method of the invention.

According to a yet further aspect of the invention there is provided use of the methods of the present invention in producing a radiolabelled biomolecule.

According to a yet further aspect of the present invention there is provided a composition comprising a metal transport protein preferably an iron transport protein such as lactoferrin coupled to a chemotherapeutic agent.

Preferably the lactoferrin is radiolabelled and more preferably is radiolabelled with technetium.

Preferably, the composition may be lyophilised; it may additionally or alternatively include an appropriate excipient, carrier or diluent.

According to a yet further aspect of the invention there is provided a pharmaceutical comprising lactoferrin or radio-labelled lactoferrin coupled to a chemotherapeutic agent.

Preferably the radio-label is technetium.

The drug conjugates or compositions comprising lactoferrin coupled to a chemotherapeutic agent of the present invention are effective for the usual purposes for which the corresponding drugs are effective, and have superior efficacy because of the ability, inherent in the complex, to transport the drug to the desired cell where it is of particular benefit. Further, because the conjugates/compositions of the invention can be used for modifying a given biological response, the drug moiety is not to be construed as limited to classical chemical therapeutic agents. For example, the drug moiety may be a protein or polypeptide possessing a desired biological activity. Such proteins may include, for example, a protein such as tumor necrosis factor. The preferred drugs for use in the present invention are cytotoxic drugs, particularly those which are used for cancer therapy. Such drugs include, in general, DNA damaging agents, anti-metabolites, natural products and their analogs. Preferred classes of cytotoxic agents include, for example, the enzyme inhibitors such as dihydrofolate reductase inhibitors, and thymidylate synthase inhibitors, DNA intercalators, DNA cleavers, topoisomerase inhibitors, the anthracycline family of drugs, the vinca drugs, the mitomycins, the bleomycins, the cytotoxic nucleosides, the pteridine family of drugs, diynenes, the podophyllotoxins, differentiation inducers, and taxols. Particularly useful members of those classes include, for example, methotrexate, methopterin, dichloromethotrexate, 5-fluorouracil, 6-mercaptopurine, cytosine arabinoside, melphalan, leurosine, leurosideine, actinomycin, bleomycin, cis-platin, daunorubicin, doxorubicin, mitomycin C, mitomycin A, carninomycin, aminopterin, tallysomycin, podophyllotoxin and podophyllotoxin derivatives such as etoposide or etoposide phosphate, vinblastine, vincristine, vindesine, taxol, taxotere retinoic acid, butyric acid, N.sup.8-acetyl spermidine, camptothecin, and their analogues and metal ions.

We have found that metal transport proteins such as transferrin or lactoferrin provides an alternative general transport protein for chemotherapeutic agents. We have already demonstrated that lactoferrin is specifically taken up by tumours (PCT/GB01/03531). We now show that transferrin and/or lactoferrin coupled to a chemotherapeutic agent, for example and without limitation taxol, cis-platin, bloemycin, daunorubicin, metal ions such as titanium and such like enhances the uptake of these chemotherapeutic agents into tumour sites in vivo.

According to a yet further aspect of the invention there is provided use of transferrin or lactoferrin coupled to a chemotherapeutic agent and optionally radiolabelled for the treatment of cancer. The invention also provides a method of treatment comprising administering a therapeutically effective amount of transferrin or lactoferrin coupled to a chemotherapeutic agent to a patient requiring treatment for cancer.

According to a yet further aspect of the invention there is provided use of transferrin or lactoferrin coupled to a chemotherapeutic agent and optionally radiolabelled for the manufacture of a medicament for the treatment of cancer.

It will be appreciated that lactoferrin coupled to a chemotherapeutic agent may also be labelled with a radionuclide. Preferably, the radionuclide is technetium and preferably the lactoferrin is radiolabelled as hereinbefore described with the additional component of a chemotherapeutic agent.

According to a yet further aspect of the invention there is provided a method of diagnosing the presence of a tumour comprising administering a technetium labelled transferrin or lactoferrin product produced by the method of the present invention to a patient suspected of, or having, a tumour and imaging the presence of the labelled product in the body.

It will be appreciated that the technetium labelled product may be made prior to or during the diagnostic investigation. Therefore the product may be made either in a laboratory or in the hospital environment.

According to a yet further aspect of the invention there is provided a method of treating a patient having a tumour comprising administering a therapeutically effective amount of a composition comprising a chemotherapeutic or gene therapy agent coupled to a technetium labelled transferrin or lactoferrin product produced by the method of the present invention.

Preferably, the composition is administered repeatedly over an appropriate dosing regimen and may be administered by i.v, i.m., sub-cutaneous, oral route or may be administered directly to the tumour site or by any other route deemed appropriate.

Preferably, the composition is prepared prior to or at the time of therapy.

The invention will now be described by way of example only with reference to the following Figures wherein:

FIG. 1 illustrates Tc-99m labelled lactoferrin binding and uptake by MCF7 breast tumour cells.

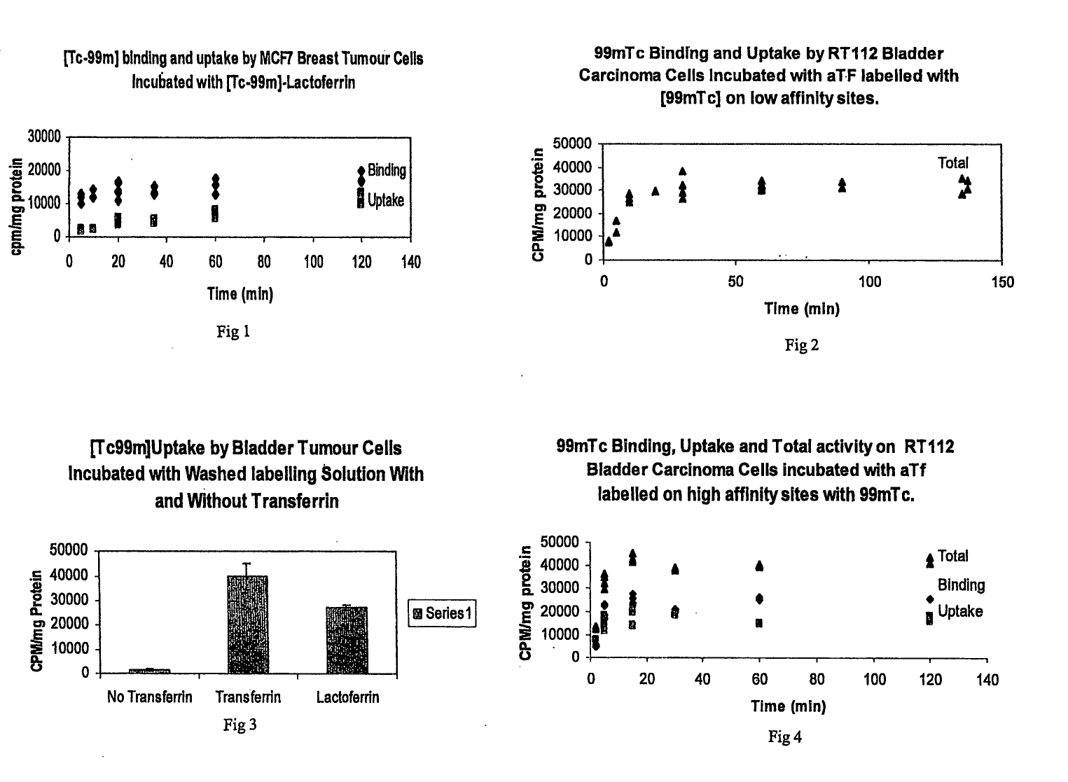

FIG. 2 illustrates Tc-99m labelled apotransferrin binding and uptake by RT112 bladder carcinoma cells.

FIG. 3 illustrates Tc-99m uptake by RT112 bladder carcinoma cells incubated with labelling solution with and without transferrin

FIG. 4 illustrates Tc-99m binding, uptake and total activity in RT112 bladder carcinoma cells incubated with aTf labelled on high affnity sites.

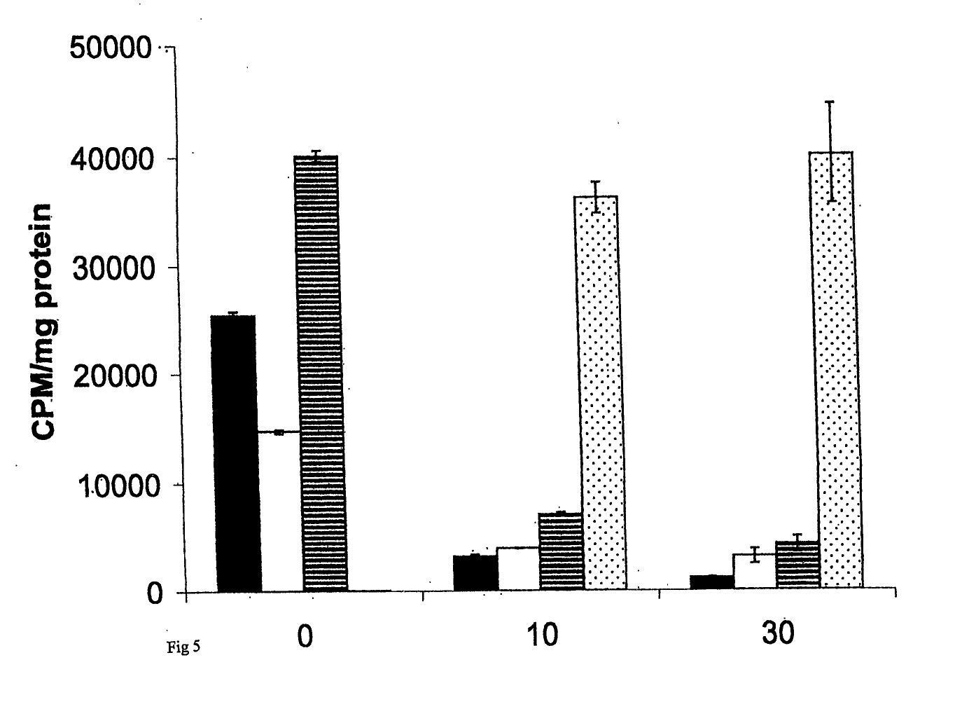

FIG. 5 illustrates ^{99m}Tc uptake by RT112 incubated for 60 min in the presence of sTf labelled on high-affinity sites then after a further 10 and 30 min incubated with unlabelled sTf. (Externally bound (solid black); internalized (white); total uptake (horizontal lines); activity in medium (dots)). (Results: Mean±SD of triplicates)

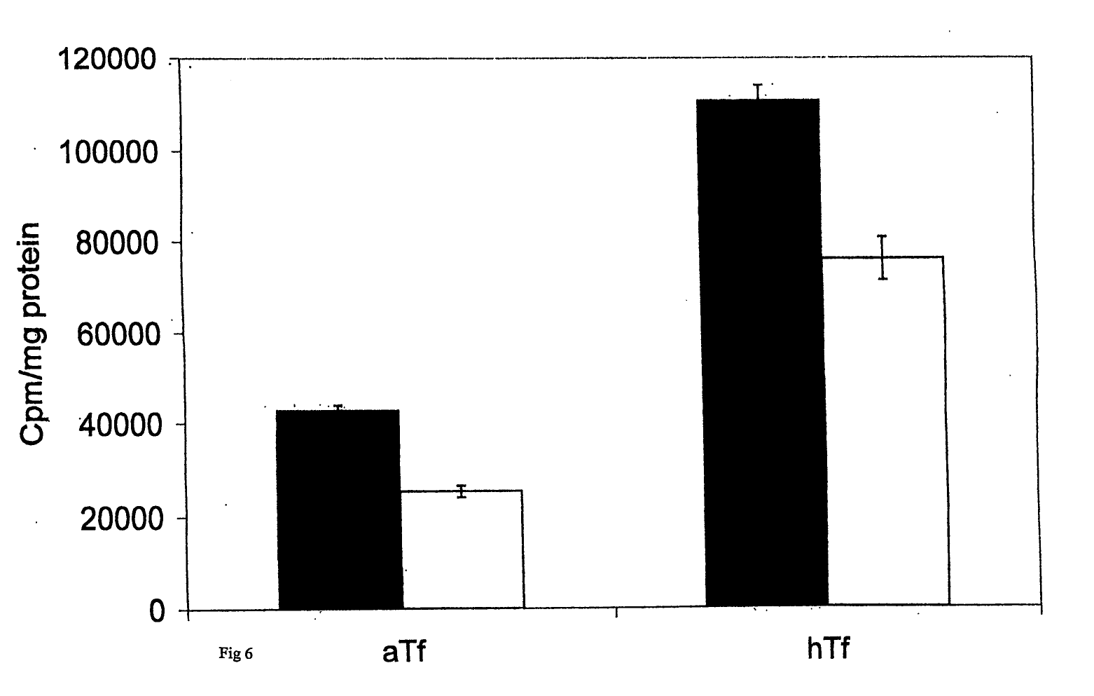

FIG. 6 illustrates ^{99m}Tc uptake (externally bound (solid black) and internalized (white)) by RT112 incubated for 60 min in the presence of apo- or holo-sTf labelled on high-affinity sites. (Results: Mean±SD of triplicates)

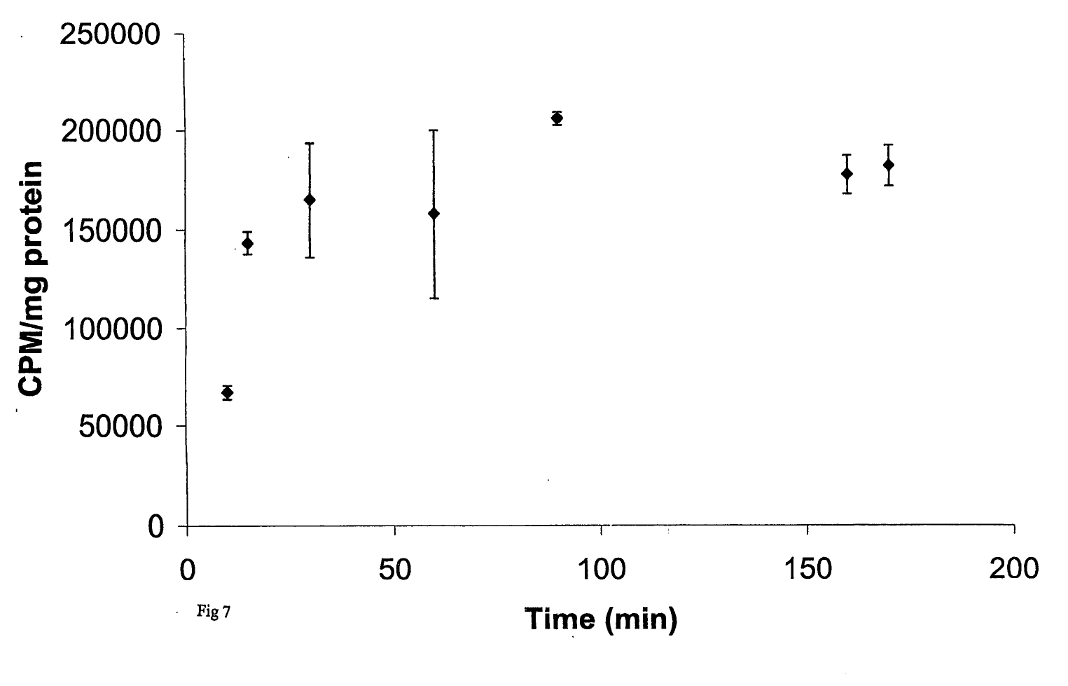

FIG. 7. Cell-associated ^{99m}Tc activity at different times of incubation of MCF7 cells with ^{99m}Tc-transferrin complex.

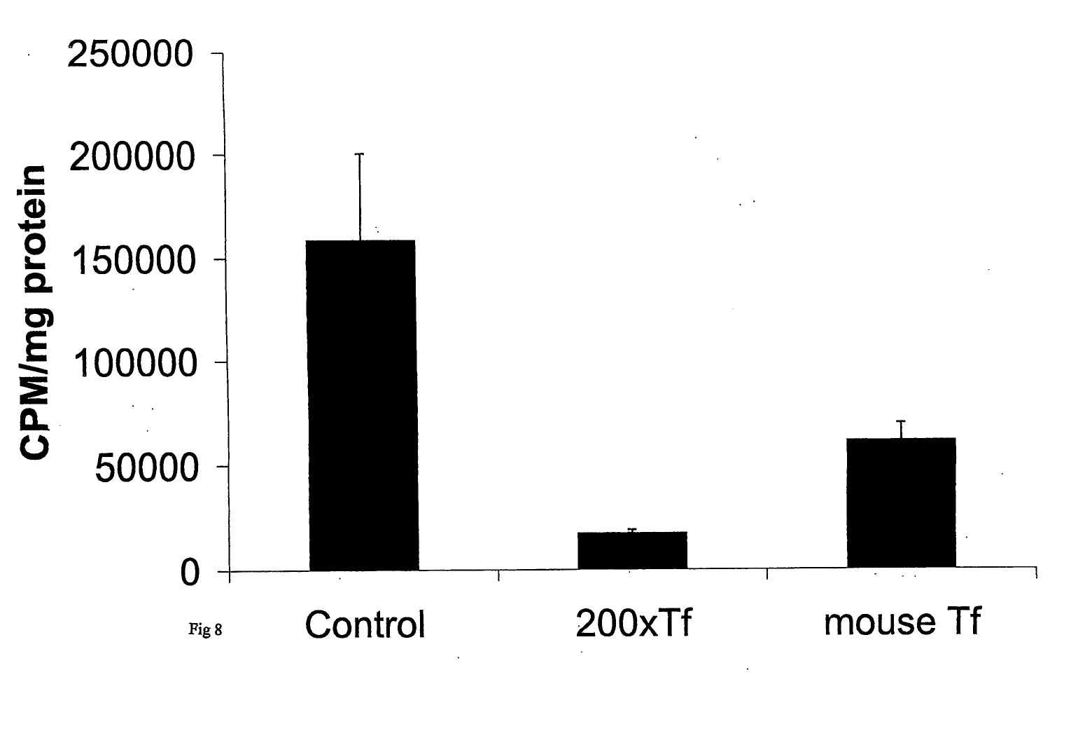

FIG. 8. Cell-associated ^{99m}Tc activity by MCF7 cells incubated with ^{99m}Tc-human transferrin complex, without and with a 200 fold higher concentration of uncomplexed transferrin, or with ^{99m}Tc-mouse transferrin complex.

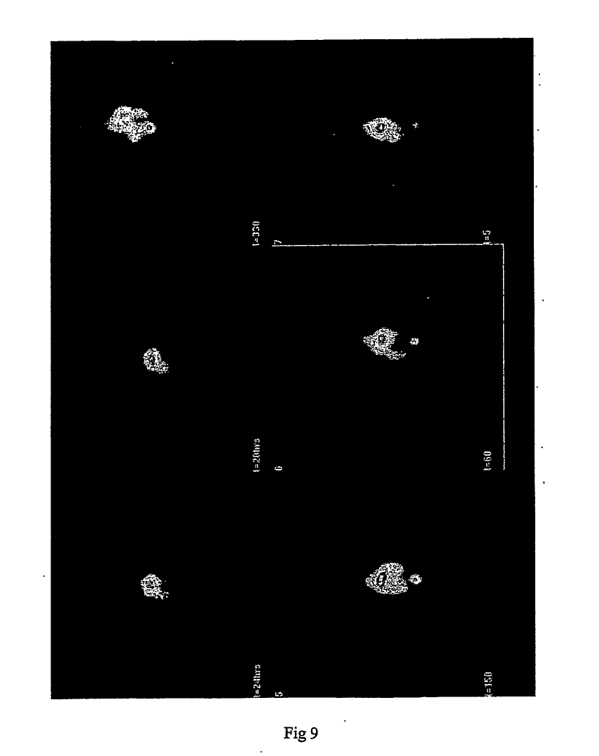

FIG. 9. Whole-body images from a xenografted mouse at different time points after administration of ^{99m}Tc-transferrin.

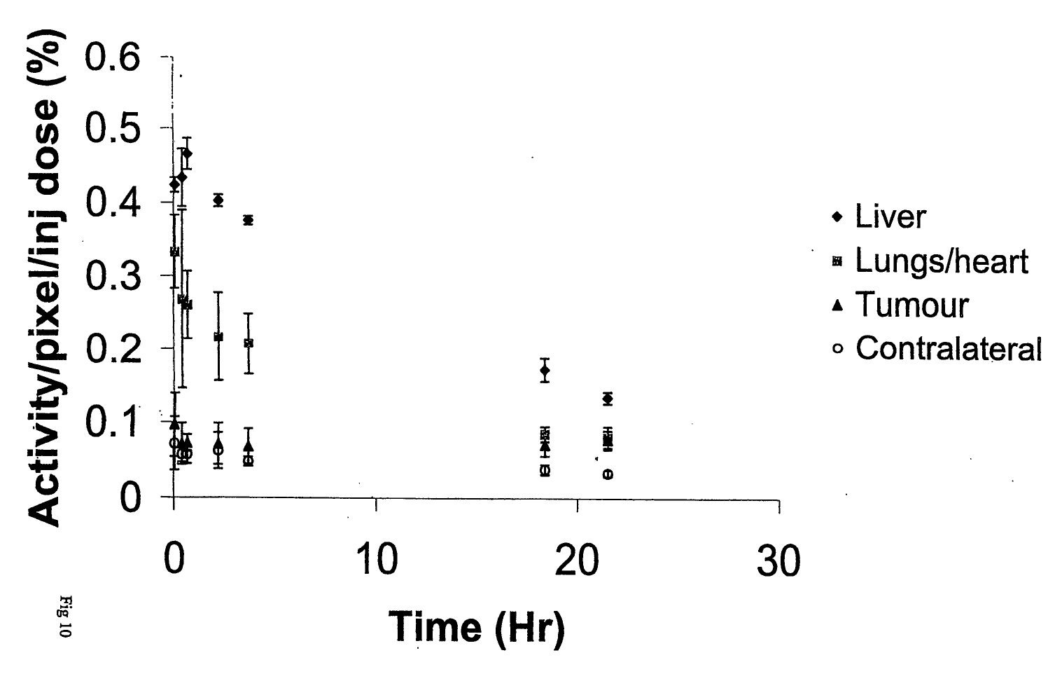

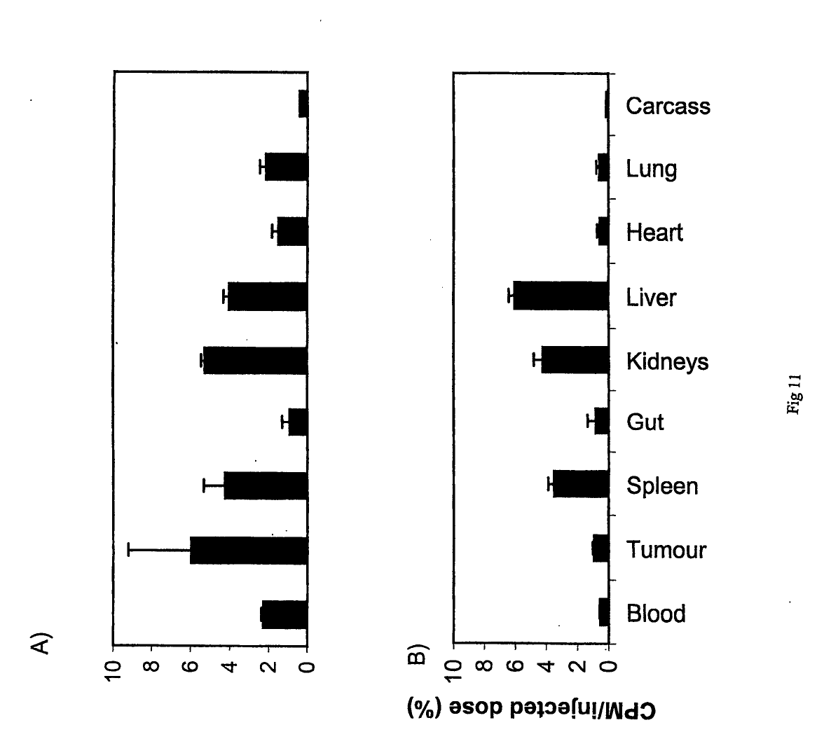

FIG. 10. Radioactivity distribution for regions over liver, lung and heart, tumour and a region contra-lateral to the tumour. Data expressed as % injected activity (total body activity at first time point) corrected for physical decay.

FIG. 11. Bio-distribution of activity in organs, blood and tumor dissected 24 h after administration of ^{99m}Tc-transferrin.

### DETAILED DESCRIPTION OF THE INVENTION

Materials and Method

Proteins were labelled in one example by incubating the protein (see concentrations below) with pertechnetate (ca. 2.3 nM), 1 mM thiourea and 7.5 μm SnCl₂ at pH 7.0 for between 0.5 to 2 hours. After incubation, the solutions are passed through a size-exclusion filter for example a Centricon 30 filter obtainable from Fisher Scientific, and subjected to centrifugation at 3200 rpm. Samples are then washed with approximately 2.5 mL PBS solution at pH 7. The filters are reversed by being turned upside down in the centrifuge and the residue on the filter is then washed out. The protein is recovered by centrifugation at 2500 rpm.

Cell Culture RT112

Binding and uptake studies were carried out on the fast growing human bladder carcinoma cell line, RT112, maintained in Dulbecco's Modified Eagle Medium supplemented with 5% Foetal Bovine Serum, 10,000 units/ml of penicillin/streptomycin. Cells were maintained in 75 cm² tissue culture flasks and sub-cultured (1:20) 4 d prior to an experiment into 25 cm² flasks. The cells were confluent at the time of each experiment.

Cell Culture MCF7

Uptake studies were carried out on the breast tumour cell line, MCF7, maintained in Dulbecco's Modified Eagle Medium supplemented with 10% Foetal Bovine Serum, 10,000 units/ml of penicillin/streptomycin. Cells were maintained in 75 cm² tissue culture flasks and sub-cultured (1:20) 4 d prior to an experiment into 25 cm² flasks. The cells were confluent at the time of each experiment.

Pre-reduction and Radiolabelling of Transferrin

The lyophilised human serum transferrin (Sigma-Aldrich, Poole UK) was dissolved in 10 mmol dm⁻³ phosphate buffered saline (PBS) and the sTf concentration determined by measuring its absorbance at 280 nm. At this wavelength sTf (serum transferrin) has an extinction coefficient of 93,000 dm³ mol⁻¹ cm⁻¹. Pre-reduction: Transferrin was reduced with 2-mercaptoethanol (2-ME) by incubating the required concentration of transferrin with 2-ME in a total volume of 0.2 ml for 25min at 20° C. prior to labelling.

Radiolabelling

All solutions for radiolabelling were made up in 10 nmol dm⁻³ PBS. The following were added to a 1 ml glass sample vial: 25 μl of a 2.8×10⁻⁷ mol dm⁻³ solution of thiourea (final concentration 1.5 mmol dm⁻¹), 25 μl of a 10⁻¹⁰ mol dm⁻³ solution of SnCl₂ (final concentration 8×10⁻⁷ mol dm⁻¹), 50 μl of pertechnetate (250 MBq ml⁻¹) obtained from a ^{99m}Tc generator and 25 μl of transferrin. The concentration of transferrin used was varied to examine the effect of sTf concentration on labelling efficiency. For the cell uptake experiments using pre-reduced and non-reduced sTf, final concentrations of 2.5×10⁻⁶ mol dm⁻¹ and 2.5×10⁻⁷ mol dm⁻¹ respectively were used. The solution was mixed and incubated for 60 min at 37° C. after which 0.85 ml PBS was added and the solution left for a further 15 min at 37° C. The labelling solution was then applied to a Centricon YD30 filter Fisher Scientific, UK) which has a molecular weight cut off of 30 kDa. The protein was washed with 1 ml and 0.50 ml of PBS then recovered into 1 ml of Medium199 (HEPES modification was used throughout). A 25 μl sample of both filtrate and of the recovered protein was counted on a gamma counter using a window of 90-180 keV.

Labelling efficiency was determined by comparing the activity recovered from the Centricon filter with the total activity used in the radiolabelling solution based on a standard made from the original pertechnetate solution. Thin layer chromatography on silica gel using saline as eluent was used to ensure that pertechnetate reduction had occurred.

Cell Uptake

^{99m}Tc-sTr Uptake: RT112 cells were washed with 3×5 ml PBS. The recovered transferrin from the filter (see ‘radiolabelling’) was added to 50 ml of Medium 199 and 4 ml added per flask of cells. Incubations were carried out at 37° C. Following incubation the cells were washed 5 times with PBS then trypsinised by the addition of 1 ml of trypsin and incubation at 37° C. for 10 min. In some experiments the internalized activity was separated from the surface bound activity by centrifuging cells at 3000 g for 5 min. The supernatant (externally bound) and after addition of 1 ml of 0.5 mol dm⁻³ NaOH, the pellet (internalized), were counted.

Displacement of ^{99m}Tc-sTr with sTr

Nine flasks of RT112 cells were incubated for 60 min with ^{99m}Tc-labelled sTr at a concentration of 7×10⁻¹² mol ml⁻¹ in Medium199 then washed 5 times with PBS. Bound and internalized ^{99m}Tc activity was determined in cells from 3 flasks as described above. The remaining flasks were incubated in triplicate for 10 and 30 min in Medium199 and 8×10⁻⁹ mol ml⁻¹ of unlabelled holo-sTr to displace the labelled sTr that was specifically bound to transferrin receptors. ^{99m}Tc activity in the medium and still present in the cells was then determined.

Protein Content

Protein content was determined by the Bicinchoninic acid method using a kit (Sigma-Aldrich, Poole UK). Cells were first dissolved overnight using 0.5 mol dm⁻³ NaOH. The solution was then neutralized using 2 mol dm⁻³ HCl as NaOH at concentrations greater than 0.1 mol dm⁻³ interfere with the protein assay.

Radiolabelling

The percentage of ^{99m}Tc bound to sTr under different labelling conditions is shown in Table 1. Labelling efficiency was found to increase with sTr concentration for both the non-pre-reduced and reduced protein. In the latter case using a ratio of 2-ME to sTr of 200 to reduce the protein, a yield of 58% was achieved using 30 μmol dm⁻³ sTr (the maximum concentration used) and about 6% using a concentration of 3 μmol dm⁻³. Lower ratios of 2-ME to sTr resulted in lower yields of labelled product. The inclusion of 1 mmol dm⁻³ DTPA in the radiolabelling formulation resulted in almost no labelling.

The stability of sTr labelled with technetium with and without pre-reduction is shown in Table 2. Without prior reduction, the ^{99m}Tc binding to sTr was labile with about 50% of its label being lost during 60 min incubation in Medium 199. In contrast 83% of ^{99m}Tc was still complexed to pre-reduced ^{99m}Tc-labelled sTr even after 21 hr at 37° C. in Medium199.

Cell Uptake; ^{99m}Tc-sTr Uptake:

MCF7 cells were washed with 3×5 ml PBS. The recovered transferrin from the filter (see ‘radiolabelling’) was added to Medium 199 to give an activity of 37 kBq/ml and 4 ml added per flask of cells. Incubations were carried out at 37° C. Following incubation the cells were washed 5 times with PBS then trypsinised by the addition of 1 ml of trypsin and incubation at 37° C. for 10 min. 0.1 ml of 5.5 mol dm⁻³ NaOH was then added to dissolve the cells and the suspension counted.

Tissue Distribution Studies

All animal experimentation was carried out under the UK's Home Office guidelines for animal experimentation. The following experiment was carried out twice on separate occasions: MCF7 cells were implanted into the left flank of 3 athymic nude mice each weighing about 20 g. In the first experiment (group 1) the tumours grew to about 2 mm in size and in the second (group 2) about 1 cm in size at the time of imaging. Each animal received of 10 MBz of the ^{99m}Tc-complex through a tail vein. A standard of the same volume as that injected was also prepared.

Immediately after injection and at different time point up to 24 h post-injection the animals were imaged using an animal-dedicated γ-camera (manufacturer). Regions were placed over the whole body, chest, liver, tumour and an area contra-lateral to the tumour. Dissection was then carried out at the end of the study to determine the bio-distribution of the complex. The samples were counted along with the standard.

### EXAMPLE 1

Apolactoferrin (aLf) was preincubated before use overnight at 4° C. with 35 μM 2-mercaptoethanol then labelled using 1.6 μM of protein labelled with 7% efficiency. The labelled protein was repeatedly washed and it was found that 92% of the label was still attached after 60 min incubation in PBS. Results are shown in Table 3.

### EXAMPLE 2

Apotransferrin (aTf) was preincubated before use overnight at 4° C. with 2-mercaptoethanol (2-ME) used at varying concentrations 2.6 μM, 12 μM and 26 μM giving labelling efficiencies of 8%, 25% and 60% respectively. The lowest concentration of 2-ME was repeated with resultant labelling efficiencies of 7% and 8.5%. After washing and 90 min incubation in PBS at 37° C., 97% of the highest labelling concentration of aTf still remained on the protein. Results are shown in Table 3.

### EXAMPLE 3

DTPA is exemplary of prior art chelating moities and forms stable chelates with a variety of metals. The amount of label on aTf dropped following a 2 hour incubation in the presence of DTPA by some 10%, from 97% to 87%, indicating that weally bound radionuclides were removed from the biomolecule.

Incubating aTf in acidic conditions also resulted in a reducing of the amount of bound radionuclide. The amount of label on the protein dropped by some 23% from 97% to 75% at pH5 after a 2 hour incubation, see Table 4.

### EXAMPLE 4

Tumour cells readily take up and bind radionuclide labelled transferrin and lactoferrin. FIGS. 1 to 4 illustrate the binding and uptake of Tc-99m labelled transferrin and lactoferrin in tumour cells. FIG. 1 shows a plot of the uptake of Tc-99m labelled lactoferrin into breast tumour cells against time, uptake is rapid and increases with time. FIG. 2 shows a plot of the uptake of Tc-99m labelled transferrin against time into bladder tumour cells. The transferrin is labelled on low affinity sites i.e. Tc is bound all over the protein. The plot shows rapid uptake which reaches a plateau around 40 minutes. FIG. 4 shows a similar plot but in this instance the transferrin is labelled only on high affinity sites. With regard to FIG. 3 there is shown bar chart of the uptake of Tc in bladder tumour cells in the absence of lactoferrin or transferrin and in the presence of either transferrin or lactoferrin. It can be seen that both proteins increase the uptake of To into tumour cells.

### EXAMPLE 5

The incorporation of ^{99m}Tc with time by RT112 cells incubated in the presence of labelled sTr is shown in FIG. 2. Both sTr labelled without (FIG. 2) and with (FIG. 4) pre-reduction show a rapid initial rate of uptake reaching a plateau at about 20-30 min after which there was no appreciable increase in ^{99m}Tc incorporation.

### EXAMPLE 6

RT112 cells that had been incubated with ^{99m}Tc-labelled sTr for 60 min and washed to remove unbound ^{99m}Tc-sTr which were then incubated in a 1000 fold excess of cold sTr lost most of their ^{99m}Tc associated activity by 10 min with a concomitant increase in activity in the medium (FIG. 5).

### EXAMPLE 7

Comparison of the activity, bound and internalized, by cells incubated for 60 min with pre-reduced apo- and holo-sTr is shown in FIG. 6. Although the labeling efficiency for the apo-sTr (29%) and holo-sTr (36%) are similar, both the bound (42,774±1228 and 110481±3298 respectively) and internalized (25240±838 and 75,990±4594 respectively) ^{99m}Tc activity is greater for the holo-protein than the apo-protein by almost a factor of 3.

### EXAMPLE 8

In common with RT112 human bladder cells, ^{99m}Tc-uptake by MCF7 tumour cells, incubated with the complex, increased rapidly for the first 30 min then reached a plateau after which no further uptake was evident for up to the 160 min (final) time point (FIG. 7).

### EXAMPLE 9

Incubation of cells with the complex and a 200 fold excess of hTf resulted in a reduction in the uptake of ^{99m}Tc to less than 12% of that of cells incubated with only the radio-labelled complex (FIG. 8). This suggests that most activity entered the cell via the transferrin-receptor. FIG. 8 also shows that the uptake of mouse Tf, labelled under identical conditions to hTf, by MCF7 cells. The uptake of the former is about 40% of the latter showing that mouse Tf does have some affinity for the human transferrin receptor.

### EXAMPLE 10

FIG. 9 shows the bio-distribution of radioactivity in one mouse at different time points after administration of the ^{99m}Tc-hTf complex. The uptake in the regions of interest shown, expressed as % cpm/injected dose decay-corrected, were similar for both groups of animals. This data from group1 is shown in FIG. 10. During the first 5 min there is high activity in the liver and lung/heart region the latter reflecting blood activity. At later time points the activity diminishes in these regions. The activity in the tumour region was about 1% of injected dose and remained so for the duration of the study whilst activity in the region contra-lateral to the tumour decreased from about 1.5% to 0.5% of injected activity. The tumour/Blood ratio increased to 2.4 at 21 h. Data for the second group of mice showed similar trends.

### EXAMPLE 11

FIG. 11 shows the dissection;data from the two groups of mice, 24 h after injection, expressed as % cpm/injected dose. The distribution of activity in normal tissues in the two groups is very similar although the uptake of complex by tumors in the first group appears to be higher than in the second group. This is not statistically significant due to the large standard deviation of tumor uptake by the small tumors. The means of the tumor/blood ratios is 2.7 and 1.75 in the first and second groups respectively.

To check for colloid formation the radiolabelled product was passed through a 100 kDa filter. All the activity was found to pass through the filter.

The present invention has shown that human serum transferrin labelled on high affinity binding sites by pre-treating it with 2-mercaptoethanol and using thiourea as an exchange ligand followed by a filtration procedure to remove non-bound technetium provides improved purity of labelled molecule.

We have used a fully quantitative method for determining labelling efficiency. This method involved comparison of the activity retained on a thoroughly washed Centricon YD30 filter through which the radiolabelling mixture had been filtered, with the activity used in the original labelling procedure. A labelling yield of 58% was achieved using a transferrin concentration of only 30 μmol dm⁻³. Comparable labelling efficiencies were obtained using 2-ME to reduce albumin (62%) and IgM (55%). The specific activity of the labelled transferrin was 2,150 TBq mol⁻¹ comparable with that of a study in which polypeptides at a concentration of 1 mmol dm⁻³ were labelled with technetium producing a specific activity of 2,000 TBq mol⁻¹.

The sTf molecule possesses a total of 8 disulfide bridges, formed from 16 cysteine residues. Pre-treatment with strong reducing agents, such as 2-ME, may possibly open up disulphide bridges resulting in two reduced sulfide sites to which ^{99m}Tc can bind. To achieve a high labelling yield, a ratio of 2-ME:transferrin of about 200 was required. Using a ratio of about 20 still produced labelling as long as the concentration of 2-ME was about 8 mmol dm⁻³. Interestingly 0.8 mmol dm⁻³ 2-ME completely abolished all labeling including that of low affinity sites.

Our cell studies have shown that there is an initial rapid rate of uptake of ^{99m}Tc that reached a plateau after about 20 min. A very similar time-uptake curve was found for the uptake by human histiocytic lymphoma cells of sTf labelled with ¹²⁵I and ¹⁸F. This uptake behaviour suggests that the pre-reduction step did not diminish sTf's ability to bind to its receptor. The curve corresponds to a rapid binding and internalization of activity until the sTf has achieved saturation

The binding and uptake of ^{99m}Tc-labelled holo-sTf by RT112 cells over a 60 min period was found to be higher than that achieved by apo-sTf. However, since the affinity of sTf receptors for apo-sTf is similar to that of holo-sTf and that their expression is similar, it is possible that the cell may recycle apo-sTf more rapidly than holo-sTf as the former is free of iron and so of no use cell. The results suggest that for any imaging purposes the holo-form should preferably be used.

In this study we also attempted to label sTf in the Fe³⁺ binding site by not pre-reducing the protein. Under these labelling conditions 50% of the label was lost after 2 hours in Medium 199. The rate of loss of ^{99m}Tc from sTr was more rapid at pH5. Cell uptake studies showed that, in contrast to sTf labelled with Fe in the Fe³⁺ binding site where a continuous increase in ⁵⁹Fe activity occurs, there was no apparent accumulation of ^{99m}Tc with time after the saturation of binding sites with sTf had occurred.

Without pre-treatment with a denaturing agent and with no apparent binding to the Fe³⁺-binding site, the ^{99m}Tc would have become associated with low-affinity binding sites. Although sTf labelled in this way produced a similar time-activity curve to that of sTf labelled on high affinity sites, the loss of at least 50% of the activity into the blood would result in a high background so compromising its in-vivo use.

With regard to the in vivo and bio-distribution of the complex in xenografted mice, dissection data from mice killed at 24 h post-administration of complex showed mean-uptakes of 6 and 1.7% ID/g for small and large tumors respectively. This result is in sharp contrast with the data of Paik et al where only 0.36% was found to be taken up in tumours. Our studies indicate that technetium labelled transferrin prepared by the method of the present invention is a suitable imaging agent. Moreover, we believe that the method of the present invention is applicable to improving the labelling efficiency of other biomoecules that have hitherto been unsuitable candidates due to poor labelling efficiency or have yet to be manufactured.

The in vivo results showed that xenografts tend to become very necrotic as they grow beyond 1 or 2 mm in diameter due to a degree of incompatability between the mouse blood system and the vasculature of the human tumour. The higher activity associated with smaller tumors probably reflects the lower proportion of necrosis in smaller tumors.

The blood/tumor activity-uptake ratios were 2.7 and 1.75 for the small and large tumors respectively. These blood/tumour ratios are similar to those obtained by others who radio-labelled molecules that bind to other types of receptors over-expressed on tumours.

An advantage of targeting the transferrin receptor is that its over-expression is a characteristic of most if not all tumours. In common with other tracers our results do show high kidney and liver uptake.

The mechanism accounting for high liver uptake of transferrin complexes may be due to the presence of transferrin receptors on liver cells by which transferrin is removed from the circulation.

The in-vivo and ex-vivo findings of this study suggest that the ^{99m}Tc-transferrin complex could be a useful imaging agent. Improvement in tumor/blood ratio could be achieved by the administration of anti-human transferrin to reduce blood background levels.

In summary, sTf has been labelled with ^{99m}Tc to high specific activity and excellent stability by pre-treatment with 2-ME, using thiourea as an exchange ligand and a filtration step to remove ^{99m}Tc that had not complexed with the protein. The uptake of the complex by tumour cells both in vitro and in vivo is similar to that of sTf covalently-labelled with other radionuclides.

TABLE 1
Effect of apo-sTr concentration and 2-ME pretreatment incubation
concentration, and molar ratio to sTr, on labelling efficiency
using 1 mmol dm⁻³ thiourea as exchange ligand unless otherwise
stated. Yields in brackets are for results using holo-sTr.

| Pre-labelling reduction | | Labelling | | |
|---|---|---|---|---|
| [2ME] | | [STr] | | |
| (mmol dm⁻³) | 2ME:sTr | (μmol dm⁻³) | Yield (%) | Comments |
| — | — | 60 | 30 | |
| — | — | 3 | 19, 27, 32 | |
| — | — | 0.6 | 5 | |
| — | — | 0.2 | 4, 5 | |
| — | — | 0.1 | 1 | |
| 8 | 19 | 30 | 21 | |
| 85 | 188 | 30 | 58 | |
| 85 | 188 | 30 | 40 | 10 mM |
| | | | | thiourea |
| 8 | 47 | 10 | 15 | |
| 35 | 194 | 10 | 23, 34 (36) | |
| 0.8 | 24 | 3 | 0 | |
| 8 | 178 | 3 | 4, 6, 8 (8, 15) | |
| 8 | 178 | 3 | <1 | 1 mM |
| | | | | DTPA |

TABLE 2
Stability of sTr labelled with technetium
with or without 2ME pre-reduction.

| | Incubation | Incubation time | | | |
|---|---|---|---|---|---|
| Labelling | Conditions | 20 min | 60 min | 120 min | 21 hr |
| Not | Medium (pH7) | 62, 66 | 47, 53 | 50 | |
| pre-reduced | | 58, 67 | 48, 49 | | |
| | NaH₂PO₄ (pH5) | 30, 31 | 16 | | |
| Pre-reduced | Medium (pH7) | | | | 83, 82 |

TABLE 3
Labelling Efficiency (%)

| Transferrin | Concentration | Low Affinity | High Affinity Sites |
|---|---|---|---|
| type | (mM) | Sites | (Pre-treated with 2ME) |
| Serum | 0.06 | 30 | |
| | 0.03 | | 58 |
| | 0.02 | 25 | |
| | 0.01 | 17 | 23, 34* |
| | 0.003 | 19, 27, 32* | 4, 6, 8* |
| | 0.0006 | 5 | |
| | 0.0003 | 4, 5 | |
| | 0.0001 | 1 | |
| Lactoferrin | 0.3 | 29 | |
| | 0.1 | 26 | |
| | 0.01 | 26 | |
| | 0.002 | | 7 |
| | 0.001 | 14, 16, 20* | |
| | 0.0001 | 0.5 | |

*Repeats from different days.

TABLE 4
Stability(% Tc still attached to Transferrin)

| Trans- | | Condi- | Time(min) | | | |
|---|---|---|---|---|---|---|
| ferrin | Labelling | tions | 20 | 60 | 120 | 180 |
| Serum | Low | pH7 | 62, 66, | 47, 53, | 50 | |
| Tf | affinity | | 58, 67 | 48, 49 | | |
| | Low | pH5 | 30, 31 | 16 | | |
| | affinity | | | | | |
| | High | pH7 | | | 97, 97, 100 | |
| | affinity | DTPA | | | 87, 94 | |
| | | pH5 | | | 75, 88 | |
| Lacto- | Low | pH7 | | 62, 51, 68 | | 46 |
| ferrin | affinity | pH5 | | 54 | | 22 |
| | High | pH7 | | 92 | | |
| | affinity | | | | | |

1. A method of radio-labelling a biomolecule comprising contacting the biomolecule with a source of radionuclide in the presence of a weak transfer ligand wherein the weak transfer ligand is thiourea, urea or ammonia.

2. The method of claim 1 wherein the weak transfer ligand has an association constant of between 0.01 dm³ mol⁻¹ and 1000 dm³ mol⁻¹.

3. The method of claim 1 wherein the weak transfer ligand is a non-chelating, low-stability constant weak exchange ligand.

4. The method of claim 1 wherein the radionuclide source is a ^{99m}Tc source.

5. The method of claim 5 wherein the ^{99m}Tc source is pertechnetate (TcO₄⁻).

6. The method of claim 1 wherein the radionuclide source is ⁵⁷Co, ⁶⁷Cu, ⁶⁷Ga, ⁹⁰Y, ⁹⁷Ru, ¹⁶⁹Yb, ¹⁸⁶Re, ¹⁸⁸Re, ²⁰³Pb, ¹⁵³Sm, ²¹²Bi or a combination thereof.

7. The method of claim 1 further comprising using a reducing agent to convert pertechnetate (TcO₄⁻) to Tc³⁺.

8. The method of claim 7 wherein the reducing agent is a tin(II) salt, is ascorbic acid or ascorbate.

9. The method of claim 1 further comprising passing the biomolecule, radionuclide and weak transfer ligand through a filter system.

10. The method of claim 9 wherein the filter system comprises a size-exclusion filter.

11. The method of claim 1 further comprising a reverse or double filtration process comprising:
(i) introducing the reaction mixture into a container having an open end and a closed end and being provided with a substantially transverse filter, the filter being held in a transverse position with respect to the longitudinal container walls;
(ii) collecting material of a selected size on an upper surface of the filter;
(iii) reversing the filter so that material initially collected on its upper surface is then on a lower surface of the filter; and
(iv) washing the material off said lower surface of the filter and collecting said material.

12. The method of claim 11 wherein prior to collecting material of a selected size on the upper surface of the filter, the mixture is centrifuged at a speed in a range of around 2000 rpm to 5000 rpm.

13. The method of claim 11 further comprising centrifugation at a speed in a range of around 2000 rpm to 5000 rpm subsequent to (iv) washing the material off said lower surface of the filter and collecting said material.

14. The method of claim 13 wherein centrifugation is at a speed in a range between 3000 rpm to 4000 rpm.

15. The method of claim 14 wherein centrifugation is at a speed of around 3200 rpm.

16. The method of claim 1 further comprising removing any weakly bound radionuclide by the following:
(i) exposing the radio labelled biomolecule to acid conditions; or
(ii) exposing the radio labelled biomolecule to a chelating moiety.

17. The method of claim 1 wherein the biomolecule has disulphide bonds and is pre-incubated with a biomolecule reducing agent prior to exposure to the radionuclide so as to reduce disulphide bonds into two sulfhydryl bonds.

18. The method of claim 17 wherein the biomolecule reducing agent is 2-mercaptoethanol.

19. The method of claim 17 wherein the biomolecule and the biomolecule reducing agent are incubated for a time period in a range of around 6 hours to 24 hours.

20. The method of claim 18 wherein the concentration of the biomolecule reducing agent is in the region of 2 μM to 100 μM.

21. A kit comprising a biomolecule, a source of radionuclide and the weak transfer ligand of claim 1 and, optionally, a set of written instructions.

22. A radionuclide-labelled product produced by the method of claim 1.

23. (canceled)

24. The method of claim 1 wherein the biomolecule is in holo-form.

25. A product comprising a technetium-labelled iron transport protein coupled to a chemotherapeutic agent.

26. The product of claim 25 wherein the iron transport protein is lactoferrin.

27. The product of claim 25 wherein the product is (a) lyophilized and/or (b) comprises an appropriate excipient, carrier or diluent.

28. A pharmaceutical composition comprising lactoferrin, radiolabelled lactoferrin or technetium-labelled lactoferrin coupled to a chemotherapeutic agent.

29. A pharmaceutical composition comprising lactoferrin, radiolabelled lactoferrin or technetium-labelled lactoferrin coupled to a chemotherapeutic agent in a pharmaceutically acceptable excipient, carrier or diluent.

30. The product o claim 25 wherein the chemotherapeutic agent is taxol, cis-platin, bleomycin, metal ions or daunorubicin.

31. The product of claim 26 wherein the lactoferrin is labelled with a radionuclide in a method comprising contacting a biomolecule with a source of radionuclide in the presence of a weak transfer ligand wherein the weak transfer ligand is thiourea, urea or ammonia.

32. A method of diagnosing the presence of a tumor comprising administering a product comprising technetium-labelled lactoferrin to a subject suspected of having or having a tumor, and imaging the labelled product in the body.

33. The method of claim 32 wherein the subject is human.

34. The method of claim 32 wherein the product is produced or obtainable by a method comprising contacting a biomolecule with a source of radionuclide in the presence of a weak transfer ligand wherein the weak transfer ligand is thiourea, urea or ammonia.

35. A method of treating a subject suspected of having or having a tumor comprising administering a therapeutically effective amount of a composition comprising a chemotherapeutic or gene therapy agent coupled to technetium-labelled transferrin or technetium-labelled lactoferrin.

36. The method of claim 34 wherein the subject is human.

37. The method of claim 35 wherein the composition is administered as a single dose or repeatedly by oral administration, intravenous injection, intramuscular injection, subcutaneous injection, an injection directly to the tumor site or a combination thereof.

38. The method of claim 8 wherein the tin(II) salt is chloride, nitrite or sulphite.

39. The method of claim 16 wherein the acid condition is an environment of pH 5.

40. The method of claim 16 wherein the chelating moiety is diethylenetriaminepenta-acetic acid (DTPA).
